(19) United States
(12) Patent Application Publication
(10) Pub. No.: US 2007/0282362 A1
(43) Pub. Date: Dec. 6, 2007

(54) Sampler Device

(76) Inventors: Bjorn Berg, Slanvagen (SE); Anders Tarnstrom, Hansellisgatan (SE); Olof Lindskog, Ymergatan (SE)

Correspondence Address: YOUNG & THOMPSON, 745 SOUTH 23RD STREET, 2ND FLOOR, ARLINGTON, VA, 22202, US

(21) Appl. No.: 11/547,198

(22) PCT Filed: Mar. 8, 2005
(86) PCT No.: PCT/SE05/00335

§ 371 Date: Jun. 13, 2007

(30) Foreign Application Priority Data
Mar. 30, 2004 (SE) 0400847-0

(51) Int. Cl. A61B 5/151

(52) U.S. Cl. 606/181

(57) ABSTRACT

A sampler device for extraction, collection and delivery of a blood sample, includes a lancet (50) carried by a piston (40) which is movable in a cylinder (30), a mechanism (60,70) arranged to drive the piston for expelling of the lancet out from the cylinder in the distal direction and subsequently for retraction of the piston and lancet in a proximal direction, opposite to the distal direction, the cylinder being arranged for collection of the blood sample and the piston being controllable by the mechanism to travel in the distal direction upon feeding a sample volume out from the cylinder.

### TECHNICAL FIELD OF THE INVENTION

The present invention relates to a device for extraction, collection and delivery of a blood sample.

### BACKGROUND AND PRIOR ART

In medical and health examinations small amounts of blood are generally tested. In some cases, a small but certain amount of blood is required and collected from an incision made by a lancet or needle penetrating the skin of a patient. Generally, the blood is taken at a fingertip because of the great number of blood vessels gathered at the fingertip.

In clinical tests, a predetermined amount of the blood sample is delivered to an analytical element containing chemical reagents, biochemical reagents, or immunological reagents. In point-of-care situations, devices for collection and delivery of blood samples may be included in test kits containing also the analytical means. In some applications, such as in kits for performing immunoassays, sample volumes of up to about 400 μL or even more may be required and distributed to several immunological reagents included on one general analytical element containing these reagents. Blood-tests performed on capillary whole blood in point-of-care situations is continuously developing, accompanied by demands for an acquisition of blood while causing the patient as little pain and discomfort as is possible. The accessibility of test procedures to be performed on site also raises demands for operator friendly equipment providing non-complicated and reliable operation, incorporating functions such as extraction, collection and feeding of a blood sample onto an analytical means.

A device for collecting a small amount of blood and spotting a predetermined amount of blood onto an analytical means is previously known from U.S. Pat. No. 5,569,287 (Tezuka et al). This device comprises a cylinder, a piston which is air-tightly received in the cylinder, and a puncturing tip. The puncturing tip comprises a needle contained in a blood conduit, which is disposable and arranged to be air-tightly connected to the cylinder/piston unit for expelling of the needle through the action of a sliding means activated by the descending piston and driven by a compressible spring. Blood is drawn into the blood conduit in response to a reduced pressure in the cylinder, caused by ascent of the piston. On delivery, the collected volume is discharged from the blood conduit by raising the pressure internally of the cylinder, achieved by descent of the piston. Generally, the predetermined amount of blood corresponds to the volume of the blood conduit—typically the spotted volume corresponds to the volume being sucked up into the blood conduit, which generally is less than 100 μL and preferably in the range of 5 to 50 μL. However, no means are provided in U.S. Pat. No. 5,569,287 for controlling the spotting and for feeding a preset portion or portions of the total sample volume.

### SUMMARY OF THE INVENTION

One object of the present invention is to provide a sampler device for extraction and collection of a capillary blood sample, wherein feeding of a sample volume from the device for subsequent analysis purposes is controllable.

Another object of the present invention is to provide a sampler device which is controllable for feeding a preset blood sample volume onto an analytical element.

Yet another object of the present invention is to provide a sampler device which is adjustable for distribution of preset portions of the blood sample.

Still another object of the present invention is to provide a sampler device of non-complex structure incorporating a piston/cylinder-function combining a vented operational mode for extraction and collecting, and a non-vented operational mode for feeding a sample volume out from the cylinder.

A further object of the present invention is to provide a sampler device having indicator means visible to the operator and arranged to provide a verification of complete filling of the sampler, while essentially screening the patient from the sight of blood.

Yet a further object of the present invention is to provide a sampler device of non-complex structure suitable for disposable use.

These and other objects are met in a sampler device as defined by the appended claims.

Briefly, a sampler device according to the present invention comprises a lancet carried by a piston which is movable in a cylinder. A mechanism is arranged to drive the piston for expelling of the lancet out from the cylinder in a first distal direction and subsequently for retraction of the piston and the lancet in a second proximal direction, opposite to said distal direction. The cylinder is arranged to collect the blood sample, and the piston is controllable by the mechanism for moving the piston in said distal direction upon feeding of a sample volume.

The sampler device further comprises a push-button acting on the piston in the distal direction and a return spring acting on the piston and the push-button in the proximal direction, and a housing enclosing the push-button, return spring, cylinder and piston in coaxial relation. An interlock mechanism is arranged at the inter-face between the housing and the push-button, controlling a relative movement there between, said interlock mechanism being releasable in response to a pressure applied on the push-button in the distal direction, preferably in response to a relative rotation between the push-button and the housing caused by the pressure applied in the distal direction.

The piston and cylinder preferably are arranged in combination to provide the piston a non-frictional travel length in the distal direction upon expelling of the lancet, and a friction-sealed travel length in said distal direction upon feeding of a sample volume.

The piston and cylinder are arranged in combination to provide the piston a friction-sealed travel length in the distal direction upon feeding of a sample volume, said travel length preferably being controllable for feeding of a preset volume of the blood sample.

Advantageously, the interlock mechanism is adjustable to control the travel lengths of the piston in the distal direction for feeding preset portions of the sample volume.

In one embodiment of the present invention the piston is a reflective prism, providing an indication of complete filling of the cylinder by changing its reflectivity when wetted by the liquid sample.

According to one aspect of the present invention, a disposable blood sampler having moldable components of non-complex structure comprises:
a housing;
a cylinder, integrally formed with the housing and adapted to collect a blood sample via an opening in a distal end of said cylinder;
a piston movable in the cylinder;
a lancet carried by the piston;
a push-button acting on the piston and effective for driving said piston in the distal direction for expelling of the lancet out from the cylinder;
a return spring acting on the piston and effective for retracting said piston and the lancet in a proximal direction, opposite to said distal direction;
said housing, said push-button, said return spring, said cylinder and said piston arranged in coaxial relation, and
an interlock mechanism at the interface between the housing and the push-button, controlling a relative motion there between, said mechanism being releasable in response to a relative rotation between said push-button and said housing.

### SHORT DESCRIPTION OF THE DRAWINGS

The sampler device is further explained below with reference made to the drawings, illustrating examples of the new teachings disclosed herein.

FIG. 1 shows the sampler device components before assembly, in an exploded view;

FIG. 2 shows the assembly of components in FIG. 1;

FIG. 3 is a longitudinal section through the assembly on a larger scale, showing the sampler device in a ready-to-use condition before descent of the lancet;

FIG. 4 is a sectional view transversely to a longitudinal axis of the sampler device in FIG. 2;

FIG. 5 shows the sampler device in a partially broken away perspective view, and

FIG. 6 is a longitudinal section showing a freeze position of a piston motion upon descent of a lancet incorporated in the present invention.

### DETAILED DESCRIPTION OF THE DRAWINGS

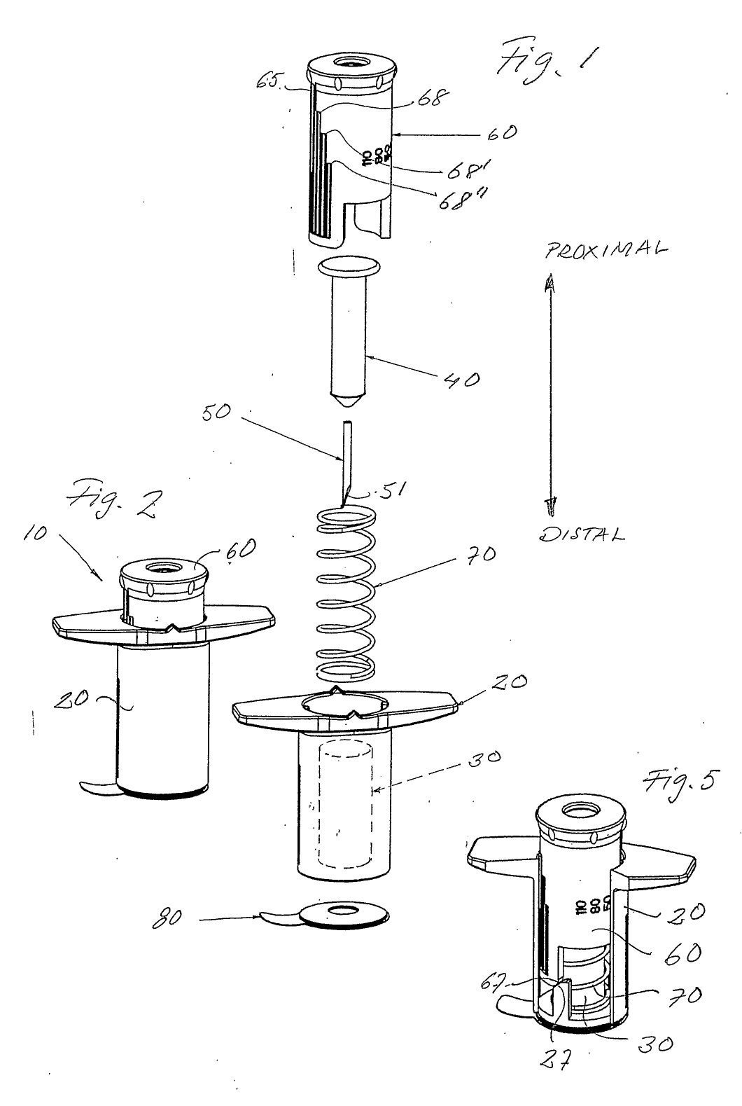

In the following description, “distal” is used for defining a first position or a first direction facing away from an operator of the sampler device, and “proximal” refers to a second, opposite position or direction facing towards the operator. In FIG. 1, the orientation is illustrated by an arrow.

With reference to FIG. 1, an embodiment of a sampler device according to the present invention comprises as its major components: a housing 20 incorporating a cylinder 30 which is carried in the housing; a piston 40, movable in the cylinder and carrying a lancet 50 in its distal end; a push-button 60 and a compressible spring 70, forming parts of a piston driving mechanism supported in the housing. A removable seal 80 preferably is attached to cover en entrance to the cylinder in the distal end of the sampler device. The assembly of these components is illustrated in FIG. 2.

Briefly explained, the sampler device operates as follows: the distal end of the housing is placed in contact with the skin; a pressure is applied to the push-button causing a piston driving force to be build up and finally to release the piston which is accelerated in the distal direction, compressing the spring and extending the lancet out from the distal end of the housing; from this distal end position, the piston and lancet are then retracted in the proximal direction by force of the spring, this way opening the cylinder to collect a volume of blood from the incision made by the tip of the lancet. When the cylinder is filled, the sampler device is removed for delivery of the blood sample by depressing the push-button, causing the piston to eject the sample volume or a part thereof via the open distal end of the cylinder.

The sampler device of the present invention is explained below to be operated by a manual pressure directly applied to the push-button. Nevertheless, the distinctive features provided may likewise be incorporated in a sampler device adapted to be operated through additional driving means, connectable to the sampler device.

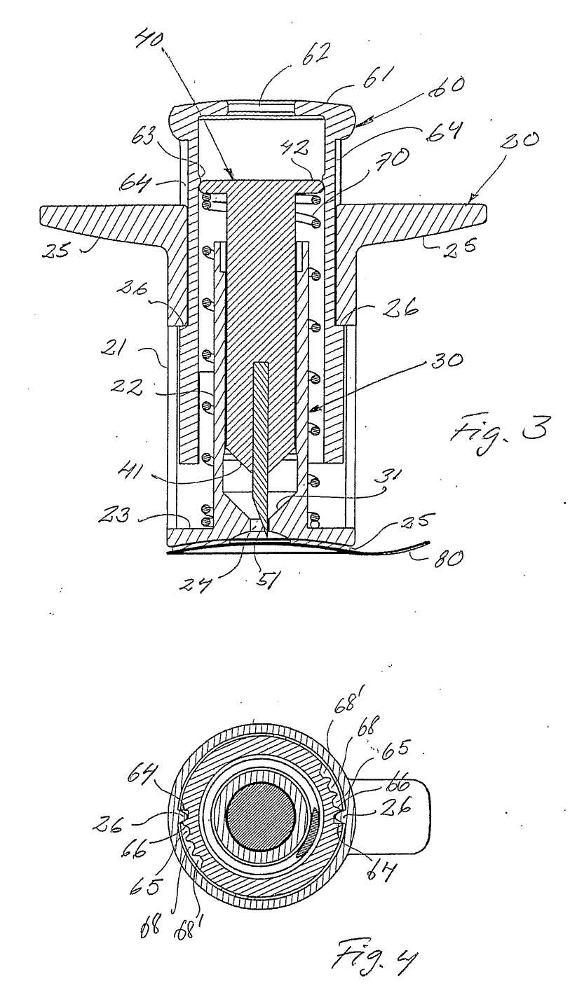

The detailed structure and operation of the sampler components of the illustrated embodiment will be more fully explained below and by reference to FIGS. 3-6. Basically, the housing 20, cylinder 30, piston 40 and push-button 60 are cylinders arranged in coaxial relation about a common central axis.

The housing 20 has an outer wall 21 radially distanced from an inner wall 22, the latter defining the cylinder 30. In the distal end, the outer and inner walls 21, 22 rise from a base 23, the base having a central through hole 24 that opens into the cylinder 30. The proximal ends of the outer and inner walls 21, 22 define the entrance of an annular space running axially towards the base 23, and formed to receive the push-button 60 and compressible spring 70 coaxially about the cylinder. Lugs 25 protrude in radial directions from the outer wall 21, providing support for the operator's fingers in use. Advantageously, the base 23 is slightly concave in its outer surface such that the periphery of the housing base provides a circumferential contact with the skin of the patient in the sampling mode . An adhesive, such as a double-coated, ring-shaped adhesive tape 25, preferably is applied in the distal end of the housing in order to provide a leak-proof adherence to the skin during collection of the blood.

The cylinder 30 has an open proximal end through which the piston 40 is inserted upon assembly. The distal end of cylinder 30 terminates in a constriction 31 connecting the inner periphery of the cylinder with the through hole 24 formed in the base 23 of the housing. The constriction 31 is shown herein as a truncated cone, other possible designs include for example a flat surface transversely to the cylinder, or a part-spherical surface. The constriction 31 operates as an end stop for the motion of the piston and lancet in the distal direction as will be further described below. In connection with immunoassays, for example, the cylinder may be dimensioned to collect a volume of up to about 400 μL or even more, typically in the range of 5-250 μL, and preferably holding a volume of at least about 100 μL.

The piston 40 is received in the cylinder and movable in the distal and proximal directions. The distal end 41 of the piston is shaped to be halted in abutting contact with the cylinder constriction 31 in the distal end position of the piston. In this position, the tip 51 of the lancet 50 protrudes from the housing base 23 at a distance which is determined for making an incision necessary to open the capillary vessels. The piston has an axial length dimensioned to reach proximally out from the cylinder in the distal end position of the piston. In its proximal end, the piston carries a shoulder 42 that reaches radially beyond the inner wall 22, above the annular space defined by the inner and outer walls 21, 22. The shoulder 42 provides a proximal support for the spring 70, acting between the shoulder and the housing base 23, and which is operative for returning the piston in proximal direction to a position where the cylinder is opened and effective for collecting a volume of blood.

The piston 40 and cylinder 30 are structured for a relative displacement in sealing contact upon feeding of the blood sample, performed through a distal motion of the piston. Yet, expelling of the lancet preferably is effected through a non-sealing or friction-free acceleration of the piston and lancet during incision, allowing evacuation of air that would otherwise be compressed by the piston moving through the cylinder.

To achieve this, as best illustrated in FIG. 6, the piston 40 has a diameter slightly less than the diameter of the cylinder and sealing is provided from a flange 43 protruding radially in the distal end of the piston. The flange 43 may be formed integrally on the piston, or a separate element mounted on the piston. In motion, the flange 43 seals against the cylinder periphery for a major length R while sealing is suspended at the end of the distal motion. The non-sealing travel is accomplished through an axial length Rᵢ of increased radius formed in the distal end of the cylinder 30. Thus, the cylinder 30 and piston 40 are structured in cooperation to provide the piston a travel comprising a first, friction-sealed travel length R and a second friction-free and non-sealing travel length Rᵢ, as viewed in the distal direction. As will be explained further on, the non-sealing length is related to the release of a piston driving force that is applied for expelling of the lancet 50.

Thus, during acceleration of the piston and lancet, the cylinder is vented by air being evacuated from the widened distal portion 32 of the cylinder, the air passing the piston flange 43 and escaping via the gap that is formed between the piston body and the cylinder periphery to be discharged from the proximal end of the cylinder.

Expelling of the lancet 50 is effected by a pressure which is applied to the push-button 60. The push-button 60 is a cylinder, running axially from an open distal end to a proximal end, the proximal end carrying a thumb-rest 61. A window 62 through the thumb-rest serves for indication purposes as will be explained later. The cylinder portion of push-button 60 encloses the shoulder 42 of the piston in close relation, coaxially about the spring 70, the cylinder 30 and the piston 40. The external cylindrical surface of the push-button 60 is received in sliding contact with the outer wall 21 of the housing 20. In FIG. 3, the sampler device is shown in a ready to use condition. In this initial position, the lancet 50 is fully retracted within the distal end by action of the compressible spring 70, the spring acting between the base 23 and the lower side of the shoulder 42 in the proximal end of the piston 40. The periphery of the shoulder 42 is engaged, on the proximal side thereof, by a heel 63 formed on the internal cylindrical surface of the push-button, the push-button 60 and piston 40 thus being biased in the proximal direction by action of the spring 70.

At the interface between the housing and the push-button, arrangements are made to effectuate expelling of the lancet by applying the necessary manual pressure to disengage a releasable interlock between the housing and the push-button. More precisely, the pushbutton is guided in the housing for movement in the proximal and distal directions. To this purpose, a protrusion formed on the housing is received to travel within a recess formed on the push-button, or vice versa. Upon relative movement in the distal direction, a constriction in said recess temporarily blocks the travel of said protrusion until sufficient pressure is applied to the push-button in the distal direction in order to force the protrusion to pass the constriction. In the illustrated embodiment, a relative rotation is required in order to disengage the interlock between the housing and the push-button, and thus for effecting acceleration of the piston and expelling of the lancet. In order to prevent jamming, two or more interlock mechanisms are advantageously arranged and spaced apart circumferentially at the circular interface between the housing and the push-button.

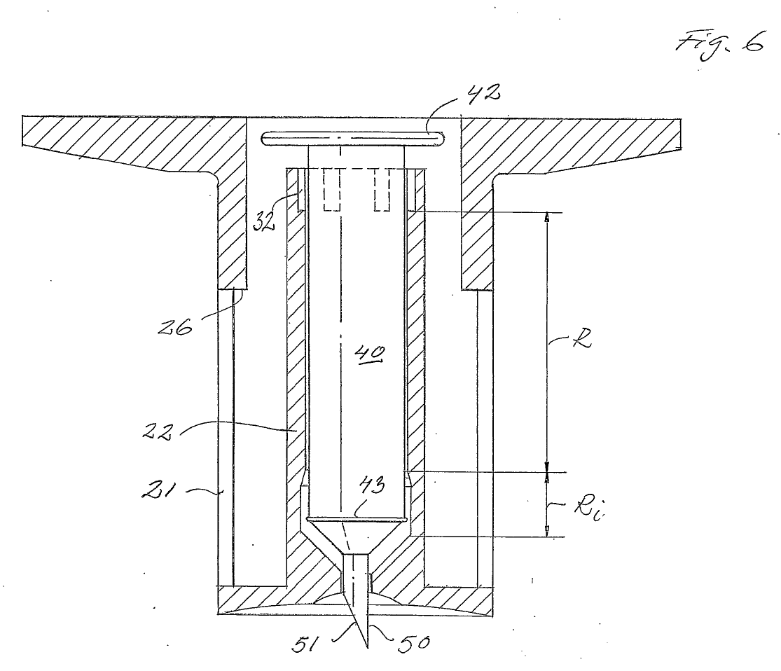

The releasable interlock mechanism is further explained by reference to FIGS. 3, 4 and 5 of the drawings. On the inner surface of the outer wall 21 of the housing, a heel 26 protrudes radially inwards to be received in a first groove 64 running axially on the outer surface of push-button 60. The groove 64 terminates in a distal end stop which is engaged by the heel 26 in the ready to use condition (see FIG. 3), wherein the push-button (and the piston 40) is biased in the proximal direction by the spring 70. A second groove 65 runs in parallel with groove 64 on the outer surface of push-button 60, the grooves 64 and 65 being separated by a partition wall 66 (see FIG. 4). The partition wall 66 and the heel 26 extend in radial direction so as to overlap by their radially outer and radially inner ends, at least one of said ends preferably being rounded or beveled in a sectional view. Upon rotation of the push-button, the heel 26 may be forced to pass the partition wall 66 in a circumferential direction, the heel leaving the groove 64 to enter the groove 65. Rotation of the push-button is accomplished through a cam arrangement involving a cam surface 27, arranged on the housing to engage a cam 67 formed on the push-button (see FIG. 5). Thus, as the push-button is depressed in the distal direction, the cam and cam surface interact for rotation of the push-button. When sufficient pressure is applied to force the heel 26 to pass the partition wall 66 and to enter into the second groove 65, the heel becomes free to travel towards the proximal end of groove 65, or in other words, the push-button is released and forced by the pressure to move in the distal direction. Beside the distinct relief in the pressure applied to the thumb-rest, the release of the push-button will be audibly observed by the operator.

The push-button pushes the piston 40 along in the distal motion, by the heel 63 engaging the periphery of piston's shoulder 42. Upon release of the interlock between push-button and housing, the piston travels the non-sealing length in the distal end of the cylinder for expelling of the lancet 50. The distal motion of the piston is abruptly terminated as the distal end 41 of the piston hits the constricted end 31 of the cylinder. At this stage, the push-button is still free to move in distal direction until the distal end of the push-button hits the base 23 of the housing. In the continued distal motion of the push-button, the manual pressure applied to the thumb-rest forces the heel 63 to pass the shoulder periphery, at least one of the shoulder or heel being rounded or beveled in a sectional view. Instantaneously, the piston is loaded by the spring 70 which returns the piston in the proximal direction until the shoulder 42 engages the lower side of the thumb-rest, thus covering the window 62. The head space internally of the push-button, determined by the axial length from lower side of thumb-rest to heel 63, is adapted for complete withdrawal of the lancet from the patient's skin as the piston is retracted by the spring 70 in the proximal direction.

Next, as the operator moves the pressure from the thumb-rest 61, the spring 70 effectuates a retraction of both piston and push-button, the piston moving the push-button further along in the proximal direction. This proximal motion is structurally terminated, such as by the heel 26 on the housing engaging an end stop in the distal end of groove 65 on the outer surface of the push-button (or by other structural elements providing a contact between the push-button and the housing, e.g.) In this motion, the piston travels the sealing length of the cylinder until the piston's flange 43 is situated in a proximal end portion of the cylinder. The cylinder 30 is thus open and ready to collect blood that flows from the incision made by the lancet to fill the cylinder from its distal end. During collection of the blood, the cylinder is vented by air being allowed to escape from the proximal end of the cylinder.

To this purpose a passage 32 is formed in a proximal portion of the cylinder periphery, said passage connecting the cylinder space with the atmosphere via an opening formed through the outer wall of the housing, e.g. The passage 32 mouths in the proximal end of the cylinder wall and extends distally beyond the piston's flange 43 in the proximal end position of the piston. One or several passages 32 may be formed as grooves running axially in the cylinder periphery, illustrated by broken lines in FIG. 6.

So far, an embodiment of the sampler device according to the invention has been explained with respect to a vented operational mode of the piston/cylinder function. Next, a non-vented operational mode will be explained by reference to FIG. 6 of the drawings.

Upon feeding of a sample volume, the piston 40 is advanced by depressing the push-button 60 in the distal direction. In this distal motion of piston and push-button, the heel 26 of the housing travels towards the proximal end of groove 65 formed on the push-button. In the feeding mode, the piston seals against the cylinder periphery by action of the flange 43, the flange frictionally engaging the periphery of the cylinder for the sealing length R thereof. Guided by the groove 65, the piston is controlled for discharge of substantially the total volume of blood collected by the cylinder.

In a preferred embodiment of the sampler device according to the present invention, the piston is controllable for feeding a preset portion of the total volume of blood collected by the cylinder. To this purpose, measures are taken to limit the travel of the piston so as to cover a preset portion of the sealing length R of the cylinder periphery upon delivery. Specifically, and illustrated in FIG. 4 of the drawings, one or several additional grooves 68, 68′ etc., are formed in parallel with the grooves 64 and 65 and separated by partition walls corresponding to the partition wall 66. The additional groove/grooves extend at individual lengths from near the distal end of the push-button towards the proximal end, each groove length permitting a corresponding distal travel of the piston upon delivery (see also FIG. 1 for a better under-standing of the arrangement of the guiding grooves). Setting the desired travel length is accomplished by turning the push-button, this way snapping the heel 26 into the subject groove, corresponding to a desired part volume of the collected blood sample. Index markings arranged on the housing and push-button provides a visual verification of the preset volume to be delivered. Obviously, the guiding grooves may alternatively be formed on the housing and the heel carried by the push-button. Accordingly, the piston is controlled by an interlocking mechanism arranged at the interface between the push-button and the housing, and controlling a relative motion there between for feeding a preset volume of the collected blood sample.

By appropriately designing the lengths of the guiding grooves, distribution of portions of the blood sample will be possible by successively setting the push-button for guidance in grooves of corresponding lengths.

In another preferred embodiment of the sampler device according to the present invention, indicator means is incorporated to provide the operator a verification of complete filling of the cylinder. In the illustrated embodiment, the indicator means involves the window 62 formed on the thumb-rest, and the piston 40. The piston 40 has a body made of plastic, or glass, or other material transparent to incident light. The light is admitted through the proximal end of the piston via the window as the operator's thumb is removed from the thumb-rest after expelling of the lancet. The distal end of the piston is prismatic and shaped to reflect the incident light, the end walls sloping at an angle relative to the longitudinal axis of the piston. Typically, the slope angle is 45°. The incident light hitting the sloping end walls is thus deflected twice and returned through the window, “lightening” the window as long as the piston end is un-wetted and exposed to air. When filling is completed, the piston end walls are wetted and covered by liquid, thus changing the reflectivity due to a change in refractory index of the medium surrounding the prism. This change will be observed by the window turning obscure. The window 62 may be an open hole through the thumb-rest, or a hole covered by a transparent material.

The lancet 50 is anchored by its proximal end in the distal end of the piston, having a sectional dimension slightly less than the diameter of through hole 23 in the housing base 23. The lancet may have a cylindrical section, the tip being shaped with one or several sharp edged facets 51 for a clean and painless incision.

All parts of the sampler device may be produced from metal or synthetic materials adapted for sterilization. A specific advantage of the illustrated embodiment is the limited number of details and the non-complicated structure of moldable components, making the sampler device specifically suitable for disposable use. Another advantage is that incision and collection of blood is essentially screened, reducing any discomfort that the sight of blood may cause the patient. Still another advantage is that the sampler device operates without applying any significant pressure to the region where the incision is made, thereby allowing an un-hindered flow of blood through the capillary vessels from where the blood is collected. Yet, a major advantage is the capacity of the sampler device according to the present invention to extract, collect and deliver desired, exact volumes of blood for subsequent analysis. Although the disclosure incorporates several advantageous features in one illustrating embodiment of the present invention, these features may be applied separately or in combination. The invention is defined by the distinctive features listed in the characterizing portion of the appended claims.

1. A sampler device for extraction and collection of a capillary blood sample, comprising a lancet carried by a piston which is movable in a cylinder, a mechanism arranged to drive the piston for expelling of the lancet out from the cylinder in a distal direction and subsequently for retraction of the piston and the lancet in a proximal direction, opposite to said distal direction, characterized by said cylinder being arranged for collection of the blood sample, and said piston being controllable by said mechanism to travel in the distal direction for feeding a sample volume out from the cylinder.

2. The sampler of claim 1, wherein the piston and cylinder are arranged in combination to provide the piston a non-frictional travel length in the distal direction upon expelling of the lancet, and a friction-sealed travel length in said distal direction upon feeding of a sample volume.

3. The sampler of claim 1, wherein the piston and cylinder are arranged in combination to provide the piston a friction-sealed travel length in the distal direction upon feeding of a sample volume, said travel length being controllable by said mechanism for feeding a preset volume of the blood sample.

4. The sampler of claim 3, wherein the mechanism is adjustable to control the travel of the piston for feeding preset portions of the blood sample.

5. The sampler of claim 1, wherein the piston is a reflective prism, providing an indication of complete filling of the cylinder by changing its reflectivity when wetted by the liquid sample.

6. The sampler of claim 1, further comprising a push-button acting on the piston in the distal direction and a return spring acting on the piston and the push-button in the proximal direction, a housing enclosing the push-button, the return spring, the cylinder and the piston in coaxial relation, wherein an interlock mechanism is arranged at the interface between the housing and the push-button, controlling a relative movement there between, said interlock mechanism being releasable in response to a pressure applied to the push-button in the distal direction.

7. The sampler device of claim 6, wherein the interlock mechanism is releasable in response to a relative rotation between the push-button and the housing, caused by the pressure applied in the distal direction.

8. The sampler of claim 7, wherein a cam arrangement is arranged at the interface between the housing and the push-button, said cam arrangement being effective for turning the push-button in response to the pressure applied in the distal direction.

9. The sampler of claim 2, wherein the piston carries a radially protruding flange in the distal end of the piston, the cylinder having an inner periphery frictionally in contact with said flange for a first friction-sealed length of the cylinder and the cylinder having an axial portion of increased radius for a second non-sealing length thereof, as viewed in the distal direction, providing a passage for air to escape in the proximal direction upon expelling of the lancet.

10. The sampler of claim 1, wherein an air passage is formed in the proximal end of the cylinder facing the proximal direction, said air passage providing ventilation of the cylinder upon collection of the blood sample.

11. The sampler of claim 3, further comprising a push-button acting on the piston in the distal direction and a return spring acting on the piston and the push-button in the proximal direction, a housing enclosing the push-button, return spring, cylinder and piston in coaxial relation, wherein an interlock mechanism is arranged at the interface between the housing and the push-button, controlling a relative movement there between, said interlock mechanism being adjustable to limit the travel of said piston in the distal direction so as to cover a desired portion of the friction-sealing length of the cylinder upon feeding of a preset sample volume.

12. The sampler of claim 11, wherein a set of parallel grooves of individual lengths are running axially on the periphery of the push-button, said grooves guiding the travel of a heel formed on the housing, said heel being received in one of said grooves in response to a relative rotation between the push-button and the housing.

13. The sampler of claim 5, wherein the piston is shaped in its distal end to reflect incident light admitted through the proximal end of the piston.

14. The sampler of claim 13, further comprising a push-button acting on the piston in the distal direction and a return spring acting on the piston and the push button in the proximal direction, a housing enclosing the push-button, return spring, cylinder and piston in coaxial relation, wherein said push-button comprises a window through which light is admitted to said piston.

15. A sampler device for disposable use, said sampler device comprising:
a housing;
a cylinder, integrally formed with the housing and adapted to collect a blood sample via an opening in a distal end of said cylinder;
a piston movable in the cylinder;
a lancet carried by the piston;
a push-button acting on the piston and effective for driving said piston in the distal direction for expelling of the lancet out from the cylinder;
a return spring acting on the piston and effective for retracting said piston and the lancet in a proximal direction, opposite to said distal direction;
said housing, said push-button, said return spring, said cylinder and said piston arranged in coaxial relation, and
an interlock mechanism at the interface between the housing and the push-button, controlling a relative motion there between, said mechanism being releasable in response to a relative rotation between said push-button and said housing.
